(19) United States
(12) Patent Application Publication
(10) Pub. No.: US 2006/0111271 A1
(43) Pub. Date: May 25, 2006

(54) Active and passive immunization against pharmacologically active hapten molecules using a synthetic carrier compound composed of similar elements

(76) Inventors: Erich Hugo Cerny, Geneva (CH); Mauel Jacques, Lausanne (CH); Michel Mpandi Michel, Lausanne (CH)

Correspondence Address: Clifford W. Browning;Suite 3700, 111 Monument Circle, Indianapolis, IN, 46204-5137, US

(21) Appl. No.: 10/997,494

(22) Filed: Nov. 24, 2004

(51) Int. Cl. A61K 38/00

(52) U.S. Cl. 514/2

(57) ABSTRACT

The present invention relates to vaccine conjugates composed of a hapten linked to a carrier compound, which are used for active or passive immunization in order to elicit antibodies against the hapten. The carrier compound used in the present invention is composed of a multitude of typically dozens of similar elements. Assuming each species of elements has at least one binding site for the hapten, this allows a) to maximize the degree of substitution of hapten molecules per carrier compound which may enhance the yield and avidity of elicited antibodies b) to use carrier compounds which are particularly easy to produce such as a polypeptide carrier leading to the manufacturing of cheap vaccines c) the production of particularly well defined conjugates, suited for rapid regulatory approval and well standardized immune responses. The hapten of this invention is a pharmacologically active molecule, or a chemical derivative or metabolite of such a molecule or any substance eliciting antibodies against such a molecule. The typical applications of the antiserum and vaccine conjugates of this invention are a) antibodies and vaccines against drugs of abuse, which are used as passive immunization in case of an intoxication or as an anti-drug vaccine such as an anti-nicotine vaccine b) further typical applications concern active or passive immunization, where one wants to modify the pharmacological activity of a drug molecule as for example to modify the half life of an AIDS medication through the interaction of specific antibodies.

### THE FIELD OF THE INVENTION

The present invention is in the fields of immunology and pharmacology, chemistry, virology and medicine and concerns the practice of medicine as well as the field of public health. The field of the present invention is more particularly immuno-pharmacology using haptens and antibodies, where the interaction between a hapten and an antibody is reversible, does not induce pathologies related to immune complex formation and can be used to alter the pharmacological characteristics of the hapten.

### BACKGROUND OF THE INVENTION

1. Historical Development of the Field

The history of modern vaccines in the field of infectious diseases has started in 1798 with Jenner's publication “An inquiry into the causes and effects of the Variolae Vaccinae”. Pasteur's work with the rabies vaccine came almost a century later (1885), but he experimented also with attenuated vaccines against anthrax, diphtheria, cholera, yellow fever and plaque. The practical application of passive immunization were blood respectively antibodies are transferred from one person to the other is based on the successful recognition of the basic blood groups A, B, 0, an achievement which was made by Landsteiner in 1901.

The first application of antibodies outside the field of active and passive immunization against infectious diseases or toxins of infectious agents was the development of Radio-Immunoassays by Yalow and Berson in 1959. The first RIA were directed against protein or peptide epitopes, which are either directly immunogenic, can be cross-linked using the glutaraldehyde for example or are easily linked to a carrier molecule through an amino or carboxylic acid functional group. The haptens used for drugs of abuse vaccines like nicotine, morphine or cocaine on the other hand need to be derivatized in order to introduce a functional group which can then be linked to the carrier protein. A significant amount of the chemistries used in today's drugs of abuse vaccines have been initially developed for use in conjugates eliciting specific anti hapten antibodies used for RIA's. In this context Spector (1) developed the first chemistry linking morphine to Bovine Serum Albumin (BSA), Langone and Van Vunakis (2) developed the first chemistry linking nicotine to a carrier protein and the first RIA for a cocaine metabolite is due to Mule (3)

The use of antibodies against digoxin in order to diminish the toxicity of the hapten in case of a digoxin overdose has been shown by Smith in 1971 (4). Berkowitz demonstrated the influence of anti morphine antibodies on the analgesic effect of morphine in an animal model mouse (5)

2. Prior Art

The first report of an active immunization against a drug of abuse with an intention to study its effect on self administration of the drug is due to Bonese et al. (6), who study heroin self administration after a vaccination against a morphine conjugate. The protective effect of the antibodies can be overcome by higher doses and the group of researchers turn to a passive immunization model of heroin self administration were they make the same observation (7). Strahilevitz (8) describes a device for removal of endogenous and exogenous haptens from the body, which is based on the use of antibodies against the hapten which should be removed and describes active and passive immunization. Kovalev et al. (9) study the effect of morphine consumption in a model where rats are actively immunized against morphine and the drug was consumed with the drinking water. These initial publications use hyper immunization protocols which lead to B cell tolerance.

The first vaccine description of a vaccine against drugs, which may induce dependence including nicotine as well as passive immunization against drugs of abuse haptens in case of an overdose is Swiss patent CH678394, “Impfstoff und Immunserum gegen Drogen” (10) filed in 1990. The field of vaccines has typically used natural carrier compounds such as Keyhole Limpet Hemocyanin or Bovine Serum Albumin (KLH and BSA) but new chemical strategies pioneered by Tam (11), Mutter (12), Tuchscherer (13) and others are based on construction of complex conjugates using an assembly of simple elements. The diploma work of Céline Nkubana published in 1999 as well the doctoral thesis of the same author published in 2002 reports on the successful use of toxins in the field of nicotine vaccines: “Vers un vaccin synthétique: syntheses de conjugues immunogeniques de derivés de la nicotine” (14), Elaboration d'un vaccin anti-nicotine: Développment et synthèse de conjugués immunogéniques de derivés de la nicotine (15)

The interaction between a specific antibody and a hapten is in principle reversible, and the duration of the interaction is determined by the avidity constant of the reaction partners Antigens with multiple binding sites for antibodies produce in the presence of antibodies classical immune complexes, were the antigen is cross linked by antibody bridges due to the fact that antibodies have at least two binding sites. These complexes produce in vivo severe pathologies known under the term immune complex induced pathologies. Haptens not producing this type of immune complex inducing pathologies are known to be of low molecular weight and to have typically only one epitope to which an antibody can attach at a given time. The specific antibodies can then be seen as hapten jugglers, which bind and release the hapten multiple times over the time course of a hapten life in vivo and which alter through this interaction the pharmacological properties of the hapten. The first descriptions of immuno-pharmaceutical compounds which are based on this principle have been described in 1994 by Cerny in two Swiss patents CH 689507, “Procédé de fabrication et tests diagnosiques relatives à des produits immuno-pharmacologiques” (16) and CH689251 “Produit destiné à la création des modifications souhaitables de la pharmacodynamique des substances pharmacologiques” (17). This concept has many applications as for example described in United States patent application 20040038871 by Fattori Daniela et al., with the title “Conjugates of amino drugs” where such vaccines with emphasis on applications in the field of oncology are described.

### BRIEF SUMMARY OF THE INVENTION

It has been possible to link pharmacologically active haptens such as nicotine to a carrier compound since the early seventies of last century and technically an anti nicotine vaccine could have been produced since this period. But the development of such vaccines had to overcome conceptual hurdles and is based on some insights belonging to different fields:

A typical antigen such as a horse serum produces in the presence of antibodies against it immune complexes which in turn produce sickness such as serum sickness and other immune complex initiated pathologies. Very small molecules having typically only one epitope for simultaneous binding by an antibody on the other hand do not lead to the formation of immune complexes which induce pathological processes in a mammal, and have an interaction with the antibody which is reversible. This phenomenon allows the use of antibodies in the presence of the hapten for therapeutic purposes.

The typical drugs of abuse vaccine such as a cocaine vaccine has to neutralize milligram quantities, whereas the typical infectious disease challenge is limited in the case of a poliovirus inocculum for example to a quantity which requires in the order of at least a billion times less antibodies. The astonishing capacity of anti drugs vaccines to interact efficiently with the real world high quantities of haptens of smokers or cocaine users is related to different challenges encountered during evolution: many germs produce toxins presenting larger antigen masses than the typical infectious inocculum. The immune system had to adapt and to learn how to churn out large quantities of antibodies when required. The system has furthermore a mechanism which keeps antibodies at a steady level in order to replace antibodies lost by regular turnover, because the half live of immune globulins is only in the order of 20 days. Experiments with plasmapheresis, high morphine or cocaine challenges or implantable nicotine pumps have shown that it is virtually impossible to deplete a mammal from its specific antibodies: a protective effect exists even after very high and repetitive hapten challenges over weeks, if the antibodies can be renewed.

The present invention relates to vaccine conjugates composed of a hapten linked to a carrier compound, which are used for active or passive immunization in order to elicit antibodies against the hapten.

The carrier compound used in the present invention is composed of a multitude of typically dozens of similar elements, leading to a conjugate with a molecular weight in excess of 10 000 Da (Dalton). Assuming each species of elements has at least one binding site for the hapten, this allows a) to maximize the degree of substitution of hapten molecules per carrier compound which may enhance the yield and avidity of elicited antibodies b) to use carrier compounds which are particularly easy to produce such as a polypeptide carrier allowing in turn the manufacturing of cheap vaccines c) the production of well defined conjugates, which are suited for speedy regulatory approval and d) well standardized immune response due to homogeneity in conjugate composition.

The hapten of this invention is a pharmacologically active molecule, a chemical derivative or metabolite of such a molecule or any substance eliciting antibodies against such a molecule. The typical applications of the antiserum and vaccine conjugates of this invention are a) antibodies and vaccines against drug of abuse, which are used as passive immunization in case of an intoxication or as an anti-drug vaccine such as an anti-nicotine vaccine b) further typical applications concern immunological treatments, where one wants to modify the pharmacological activity of a drug molecule as for example to prolong the biological half life of an AIDS medication with which specific antibodies interact.

The conjugate of the present invention is best described by the following two paragraphs:

1. A conjugate for the immunization of mammals which is able to elicit in a mammal antibodies against a given hapten, said conjugate comprising:

a) a synthetic carrier compound being composed of one or more types of similar elements, where at least one type of element has a functional group serving as a binding site for a hapten.

b) at least one hapten chosen from the group of pharmacologically active molecules, said hapten having a number of epitopes not allowing the formation of immune complexes inducing pathological changes in a mammal and being linked preferably by a covalent bond to the site of binding for a hapten of said carrier compound,

c) optionally a spacer compound, which forms a bridge between the carrier compound and the hapten and is preferably linked by a covalent bond to the binding sites of the hapten and the carrier compound.

2. The conjugate of paragraph 1 eliciting antibodies which are used for passive immunization.

Goals of the Present Invention:

It is a goal of the present invention to create vaccines for treatment against any substance, which may induce dependence (physical or psychical). Such substances are from the group comprising but not limited to: nicotine, cocaine in any form, opiates in any form, LSD (Lysergide or Lyserg Saeure Diethylamide, Merck Index 5451), PCP (phencyclidine, Merck Index 7087), amphetamine and methamphetamine, anti-depressive compounds, designer drugs, marijuana and cannabis derivatives as well as metabolites or agonists binding to the same receptors.

It is a goal of the present invention to create specific antibodies for treatment of an overdose due to any substance, which may induce dependence (physical or psychical). Such substances are from the group comprising but not limited to: nicotine, cocaine in any form, opiates in any form, LSD (Lysergide or Lyserg Saeure Diethylamide, Merck Index 5451), PCP (phencyclidine, Merck Index 7087), amphetamine and methamphetamine, anti-depressive compounds, designer drugs, marijuana and cannabis derivatives, psycho mimetic drugs as well as metabolites or agonists binding to the same receptors. The antibodies may be monoclonal antibodies or antibodies produced by genetic engineering or phage display technology. Application of such antibodies would be intramuscularly or under certain conditions intravenously, but could also by a peritoneal lavage. Antibody binding sites with a molecular weight under 40 000 Da are particularly interesting as they pass through the renal filter and will diminish rapidly the drug concentration in vivo, a strategy which has been shown to work using Fab′ fragments in digoxine intoxication (32, 33). Work with digoxin intoxicated animals has furthermore shown, that the efficiency of such preparation is not only based by the binding of the antibody to the drug molecule, but that lethal doses of digoxine for example can be ripped from the cardiac receptors by the antibody molecule (34). Fragments of antibodies with a very low molecular weight are an instance, where the elimination of the hapten may be accelerated by the antibody fragment (of a size preferably not retained by the kidneys), a feature particularly useful for drug intoxication.

It is a goal of this invention to prolong the half-life in vivo of pharmacologically active compounds in order to improve efficiency and save on costs (malaria prevention drugs such as nivaquine B, HIV enzyme inhibitors or false nucleic acid building blocks, any expensive drug). Under conditions of hapten excess the half life may be significantly prolonged: after immunization against nicotine, Hieda et al. report a more than 10 fold increase of the in vivo half-life of nicotine respectively its metabolites (29). But the relationship between the quotient of hapten/antibody concentration demonstrates an inverse exponential development and extreme retention times are achieved under conditions were the antibodies are in large excess to the hapten molecules. One of the conditions is, that the hapten does not get metabolized into a compound which is no more recognised by the specific antibody. As stated previously, antibodies may be seen as jugglers which release and rebind the hapten molecules many times depending on avidity constant, distribution volume and concentration of the two reaction partners. Very long elimination half lives are obtained, when the hapten epitope is not altered in vivo and has low serum concentrations as well as a naturally long half live. Schmidt and colleagues studied the fate of low digoxin concentrations in the presence of anti digoxin antibodies and state at the beginning of their paper: <<We now present evidence that, when injected into immunized animals, a specific hapten, namely digoxin, indeed forms complexes with its corresponding antibodies and that these complexes persist in the circulation for longer than 1 year after a single intravenous injection of that hapten.” (35) At one year the mean serum concentration of digoxin in the five BSA digoxin immunised rabbits had fallen from initially 8200 nanogram/ml to 85 nanogram/ml, which is a value comparable with the serum concentration after 12 hours of non-immunized control animals.

It is a further goal of the present invention to increase the efficiency of a pharmacologically active hapten by a phenomenon which one may call immune-concentration, which designates the fact that the hapten gets concentrated in the compartment where the specific antibodies are. In the examples below this fact is demonstrated nicely by the high nicotine concentrations in serum and the low nicotine concentrations in the brain of immunized animals after a challenge with radio-active nicotine.

It is a further goal of the present invention to obtain a lower toxicity for a pharmacologically active hapten, by changing the distribution of the hapten in the body through immunization against the hapten. Let us imagine the pharmacologically active hapten is an AIDS drug with significant toxicity for the central nervous system. We would expect lower concentrations on the other side of the blood brain barrier where the antibodies can not penetrate.

It is a further goal of the present invention to improve the efficiency of AIDS drugs. The immune-concentrations of a drug hapten in the distribution space of the specific antibodies (blood, lymph nodes, lymph fluid) should be particularly helpful for haptens with anti HIV activity.

It is a further goal of the present invention to stabilize concentrations of a pharmacologically active hapten in a mammal. Significantly less fluctuation in the concentration of a hapten is a direct consequence of the fact, that the antibodies may prolong the half life in vivo of a hapten. The nicotine vaccine model demonstrates this phenomenon too, because the nicotine concentration will less fluctuate after vaccination (own data below as well as for example Hieda et al. (29).

It is a further goal of the present invention to diminish the half-life of a given hapten in a mammal under well-defined conditions. Example 12 reports on conditions were less hapten is bound in vaccinated animals after 1 hour than in control animals. Smokeless tobacco as well as nicotine substitution products do not or only slightly increase mortality. At least for smokeless tobacco this is a surprising finding (36). The study shows life expectancy of smokeless tobacco users was shortened by only 15 days compared to non-smokers, but by 7.8 years for smokers. Vaccination against nicotine changes the pharmacodynamics of nicotine avoiding abrupt peaks at the receptor clue to the interaction of the drug with the antibodies before they arrive at the receptor. Cardiovascular morbidity and mortality represent about half of the morbidity and mortality due to tobacco and it seems reasonable to expect, that the vaccine may diminish the cardiovascular toxicity of nicotine. The same line of reasoning applies of course also to passively absorbed cigarette smoke. It is therefore a further goal of the present invention to evaluate in a mammal model to which extent the vaccine against nicotine diminishes cardiovascular mortality and morbidity under conditions of active or passive smoking.

### BRIEF DESCRIPTION OF THE DRAWINGS

FIG. 1 shows the close structural relationship as well as the similarity in electric charge distribution between the nicotine molecule, acetylcholine and the nicotine hapten spacer compound as described in this invention (trans-3′-Succinylmethylnicotine).

FIG. 2 shows the results of IgA and IgG measurements in both saliva and serum as determined by ELISA using a nicotine BSA conjugate coated to the solid phase.

FIG. 3 represents the optical density (OD) readings of serial dilutions of serum evaluated in a sandwich ELISA assay with nicotine-albumin coated to the solid phase, measuring total anti-nicotine antibodies with an enzyme labelled second anti antibody.

FIG. 4 demonstrates the development of anti nicotine specific antibodies in mice as measured by the precipitation of the anti nicotine-³H(−)-nicotine complexes by ammonium sulphate in a RIA after different intranasal (i. n.) and/or subcutaneous (s. c) immunisation schedules with 30 microgram of the conjugate (nicotine CTB Berna).

FIG. 5 shows the bolus injected into the tail vein corresponding to the equivalent of 2 cigarettes (600 ng in a mice of 20 g) in mice sacrificed five minutes after injection.

FIG. 6 shows scintillation counter data (cpm) after challenge with radiolabelled nicotine (1 minute, 5 minutes, 1 hour) of non immunized control mice (G1, all groups composed of 5 animals), mice immunized by subcutaneous immunization (G2).

### DETAILED DESCRIPTION OF THE INVENTION

Fields of Application of the Present Invention:

The vaccines and antibodies generated with the conjugates of the present invention are directed against drugs of abuse, especially nicotine, cocaine and opiates as well for the modification of properties in fields such as infectious diseases, where for example the prolongation of half life of a hapten means less cost and therefore treatment of a larger population. Drugs of abuse and infectious diseases are responsible for a large segment of the pathologies linked with diminished quality of life, morbidity and mortality. An overview of the epidemiology of diseases which may eventually be influenced by the type of treatment this invention describes as well as the geographic distribution and cost to public health providers of such pathologies can be found in the papers by Murray and Lopez (18-20). Tobacco is an important factor in the epidemiology of public health and the vaccines of the present invention are particularly attractive as an adjuvant to smoking cessation treatments (21). But the potential application of the vaccines and antibodies of the present application may also be helpful for therapies, were less drug should be used for economic concerns, where the drug should be concentrated for reasons of therapeutic efficiency or in order to minimize toxicity related side effects in vivo, to cite some non-limiting applications.

Definitions:

The following definitions are given for quick reference and to enhance the understanding of the specification and the claims but should not be understood as limits.

Adjuvant: An adjuvant modifies the chemical and physical properties of a vaccine and may enhance or modify the immune response as well as its duration or influence the composition of the antibody response subclasses elicited by the conjugate. Vaccines can be applied without or with adjuvant. Application of a vaccine without adjuvant may induce fewer side effects at the place of injection and provide a shorter window of protection. A large number of adjuvant is used in animal experimentation but only a few adjuvants are approved by the regulatory authorities for application in a human vaccine. Examples of modern adjuvant widely used in humans include aluminium hydroxide or phosphate and other mineral gels, bacterial cell wall derived products as well as emulsions such as Montanide ISA-51 or compounds containing polynucleotides such as CpG 7909.

Antibody: This term refers to molecules which have a sterical structure, which is complementary to the hapten, similar to the part of the lock which is complementary to the form of the key. This complementarity allows the antibody to bind in a highly selective manner to a specific part of the hapten, which is called an epitope. The binding between the antibody and the epitope is of non covalent nature and determined by hydrogen bridges, ionic forces and the Van der Vaals force. A classical antibody has a Y shaped form with two binding sides at the tip of the two branches. Antibodies directed against an epitope are normally produced by different clones of B-cells and called polyclonal antibodies. It is possible by techniques pioneered by Kohler and Milstein (22) to generate antibodies from only one immortalized B-cell clone, which are called monoclonal antibodies. It is furthermore possible to construct binding sites by phage display technology were one obtains very basic molecules which contain mainly the binding side. It is furthermore possible by genetic engineering to generate antibodies in animals which are humanized in the sense, that the species specific constant sequence of the antibody protein is replaced by a human sequence. All these variations of antibody production lead to antibodies which may potentially be used in passive immunization treatments as for example used in the treatment of drug overdose.

Carrier compound with polypeptide respectively protein elements: the peptide element of the present invention includes at least one amino acid with a binding site for the spacer of the hapten as for example a lysine. The amino acids may contain phosphor groups, acetyl groups, sugars or any type of meta-translational modification. The elements are typically produced by solid phase synthesis but methods of genetic engineering using expression vectors may also be used. The peptide elements are most often linked together by a peptide bond. There are no known restrictions on the tertiary structure as far as the immune answer is concerned. The molecular weight of an ideal carrier compound will typically be in the 100 000 to 1 million Da range and not less than 10 000 Da. Lipids and sugars as mono- or polymer may be part of the peptide carrier. It is also possible to attach the hapten to a sugar or lipid moiety belonging to the carrier compound.

Synthetic carrier compound: the word synthetic means here that the carrier compound does not occur in this form in nature and is the product of man. This definition excludes any natural carrier compound composed of subunits such as cholera Toxin B, which occurs in nature as a pentamer of five subunits, but would include a cholera toxin B carrier, if the units are assembled in a non natural way as for example by linking with bifunctional spacer.

Conjugate: The conjugate of the present invention comprises the following: a carrier compound being composed of one or more types of similar elements, where at least one type of element has a functional group serving as a binding site for a hapten. The elements of the carrier compounds are typically linked with each other by covalent bonds as for example the peptide bond of a peptide with a repetitive sequence of amino acids. But the bond between the elements can be of non-covalent nature based as ionic forces, hydrogen bridges, polar forces and Van der Waal forces as for example in a reconstituted aggregate of Cholera toxin B or Virus like particle subunits. The hapten is chosen from the group of pharmacologically active molecules and is linked preferably by a covalent bond to the site of binding for a hapten of said carrier compound. Any mimotope based hapten eliciting high antibody titers against a given hapten can be used to substitute it. The spacer is an optional compound, which forms as bridge between the carrier compound and the hapten, a feature which may improve specificity or antibody yield of antibodies directed against the hapten. The spacer is preferably linked by a covalent bond to the binding sites of the hapten and the carrier compound.

Enantiomers, hapten enantiomers, enantiomers in the carrier compound: many pharmacologically active compounds used as haptens may exist in different enantiomeric forms, which have often a different or no pharmacological activity. In most cases one will link to the carrier only the enantiomer which exist in nature and not attach synthetic forms, in order to avoid the production of antibodies against a molecule which does not exist in nature. But great care has to be applied in the choice of the enantiomer. Nicotine for example exists in nature only as L-nicotine but more than 10% D-nicotine, which itself is a pharmacologically active and a toxic compound, can be found after combustion at temperatures which are reached during cigarette combustion (23-25). Therefore, to immunize with the racemic mixture is justified in order to maximize the vaccine effect in this case. The selection of enantiomers is also important for the carrier compound. Enzymes encountered in most mammals have no or a diminished enzymatic activity for cutting bonds where D-amino acids are involved, a fact which may be used to prolong the half live in vivo of a carrier compound by incorporation of D-amino acids into the sequence. Great care has to be exercised because too many D-amino acids create a carrier which is indigestible and can not be processed in antigen presenting cells. The same remarks apply to sugar polymers and glycosylation.

Epitope, Epitopes not allowing the formation of immune complexes: We designate by epitope the portion of an antigen or hapten that is specifically recognized by the antibody and to which the antibody binds. The hapten of the present invention has typically only one site or very few sites to which an antibody can attach at any time. This is an important feature: multiple simultaneous antibody attachment leads to immune complex formation, complement activation and the full cascade of events leading to the pathologies linked with immune complex deposition, pathologies which are well known in the field of clinical immunology. The carrier compound of the present invention contains a multitude of epitopes against which antibodies are typically induced. Conjugates and immunization schedules are optimised in order to obtain high antibody titers against the hapten epitope.

Hapten: the term as used herein designates any compound having a pharmacological activity and a molecular weight, which prohibits induction of an immune response by the hapten itself. In view of the fact that the ideal hapten of the present invention should be of a size not allowing the simultaneous binding of more than one or only very few antibody molecule at the same time, the molecular weight will typically be less than 1000 Daltons. In order to obtain a homogenous end product, haptens are preferably derivatized using methods creating only one site for binding to the carrier compound, but this is not a strict requirement.

Immunogenicity of a carrier compound: the antibody response against a conjugate (hapten and carrier) is best, if the carrier compound does not contain well conserved epitopes. Exotic protein sequences as for example epitopes belonging to a toxin or genetically very different species (KLH, keyhole limpet hemocyanin) induce very good immune answers (26).

Immunological cross-reactivity: a low degree of cross-reactivity between two haptens in a sensitive evaluation system such as ELISA (Enzyme Linked Immune Sorbent Assay) is often seen among structurally closely related compounds such as nicotine and cotinine, a consequence of the great potential specificity of antibodies. Potential cross-reactivities have to be carefully evaluated. Anti nicotine vaccines should not elicit antibodies cross-reacting with acetylcholine which would be lethal or produce severe side effects. One would also logically like for any vaccine to have no cross-reactivity with pharmacologically inactive metabolites, which would be a waste of antibodies. If one devises an anti heroine vaccine one would like to develop a vaccine which reacts strongly with heroin but not with methadone, which is a long acting opiate receptor agonist used for substitution therapy. One would also like in the case of an anti-heroin vaccine a minimal cross-reactivity with opiate analgesics, which the patient may need later in life. If one wants to prolong the half live of an anti AIDS drug or an anti malaria drug, one is interested in antibodies directed against a drug which is not or only slowly inactivated and has a high pharmaceutical activity at low hapten concentration.

Mimotope: the term mimotope has been historically used for peptide sequences which structurally resemble an unrelated molecule and which are able to elicit cross-reactive antibodies against the unrelated molecule (27). But they can also be composed of non amino acid compounds and we will use the term in the broadest sense. Mimotopes elicit normally lower antibody titre and less specific antibodies than the original haptens, which limits their usefulness, a statement with which not all researchers agree. Mimotopes may be of particular interest when antibodies against a toxic compound as for example an allergen have to be produced (28), or a “self” epitope of the mammal against which a response is difficult to induce. Any compound which elicits an adequate antibody response against the hapten of interest may be considered as a replacement of the hapten in the synthesis of the conjugate.

Molecular weight of the conjugate, lower and upper limits: The molecular weight of the conjugate has to be at least 10 000 Dalton or more in order to elicit a significant immune response. The molecular weight of the conjugate ideally is in the order of a couple of hundred thousand Da and may be in excess of a million Da. There is a significant enzymatic activity in the extra-cellular compartment as well as inside immune-competent cells, which means that the upper molecular weight is not very important, because the natural proteins are easily cut during the preparation of the immune response.

Passive immunization and active immunization, vaccination: As used herein, the term “active immunization” refers to the induction of an immune response in a mammal with the use of a hapten-carrier conjugate which may be applied with or without an adjuvant. Passive immunization refers to the transfer of antibodies from one individual to another in order to obtain a pharmacological effect. The amount of conjugate given per immunization as well as the immunization schedule are crucial for an optimal active immunization especially in applications were the immunized mammal has to produce high quantities of antibodies of high avidity in order to be protected as for example in drugs of abuse vaccines. Amounts of conjugate which are to important lead to B-cell tolerance where the immune system stops producing the specific antibodies and to low amounts of conjugate lead to T-cell tolerance, where no antibodies at all are produced. A typical active immunization with the conjugates of the present invention contains a dose of more than 1 microgram of conjugate and less than 1 milligram of conjugate applied at least one time. The preferred schedule for active immunization with the conjugates of the present invention is a dose range between 20 to 100 micrograms of conjugate applied 2 to 4 times with a time interval of two to 4 weeks between each application. An active immunization based on the conditions as mentioned leads to a long lasting protective effect a process which we call a vaccination.

Response to immunization: The response to immunization involves the activation and proliferation of B- and T-lymphocytes starting with the conjugate being taken up and processed by antigen presenting cells. The conjugate of the present invention is normally optimized in order to obtain a high stimulation and duration of the antibody response, by procedures known to those skilled in the art (dose and adjuvant finding studies) whereby in most instances antibodies of high avidity and specificity are desired. Furthermore, the conjugates of the present invention are in general chosen to avoid any cytotoxic response or any induction of allergic side effects (as induced by antibodies of the Ig E class for example).

Requirements for the galenic preparation of the conjugate: the conjugate being used for veterinary or human application has to be sterile and endo-toxin free. European—as well as United States Federal Drug Administration approved Good Manufacturing Practices—require synthesis of the conjugate under strictly sterile conditions as well as avoidance of any environmental contamination by a controlled environment with controlled air-flow. The pH of an injectable solution is in a neutral range, may contain a buffer system and has preferably a physiologic osmotic pressure. It may contain inert stabilizers or fillers such as mannose and may be in liquid, powder or solid form. Manufacturing of galenic preparations involves typically a purification step such as dialysis in a buffer solution to get rid of excess hapten, spacer molecules or organic contaminants and sterilisation step such as sterile filtering as well as lyophylization to increase shelf life. Galenic preparations may contain sugars such as mannose or salts as well as inert fillers or inert gas and UV shielding in order to prolong shelf life.

Spacer and bond between hapten and carrier: Normally the association of the hapten with the carrier component is a covalent bond which is resistant to cleavage by in vivo conditions. Covalent bonds which are often used include peptide, amide, amine, carboxyl, hydroxyl, ester, ether, imide, aldehyde, hydrazine, diazonium, halogenide and sulphur based bonds. It is also possible to use a spacer between the hapten and the carrier compound which exposes the hapten and which may enhance the immune response.

The strategies of chemical coupling are often optimized in order to avoid any cross-linking of the carrier elements with other carrier elements, a phenomenon known from non specific linking methods. Hetero-bifunctional spacers having two different functional groups on each side of the spacer are particularly useful if one wants to avoid cross-linking of the carrier molecule. The specificity of the functional groups is chosen so that one group reacts with the hapten and the other group with one particular group of side chains or other well defined functional group of the carrier component.

Trans-dermal nicotine patch and nicotine substitution therapy: nicotine substitution helps overcome withdrawal symptoms in smokers. A smoker vaccinated against nicotine may develop full blown withdrawal symptoms because the vicious circle between drug application and instant gratification is interrupted by the binding of the nicotine to the antibody. It may be indicated in such circumstances for a limited period of time to apply a substitution therapy such as a nicotine patch, nicotine chewing gum or any other form of substitution. The vaccine respectively the specific antibodies elicited by the vaccine prolongs the half life of the nicotine molecule in the body, which should be considered when formulating such substitution products, which release nicotine over an extended period of time. Immune-concentration of nicotine and accumulation in the blood may necessitate a lower dose of nicotine during substitution therapy in vaccinated individuals. Our work and work of others (29) has shown with the help of implanted nicotine dispensing pumps a significant protective effect of the vaccine even in cases of a massive stochiometric nicotine overload (nicotine haptens divided by the number of specific antibody binding sites) which means that substitution therapy will not eliminate the vaccine protection.

Viral or bacterial coat element: virus coat elements are also known under the name virus like particles (30) and have been used for immunization purposes. Neither the viral or bacterial coat elements nor the space they may enclose should contain nucleic acid. They are therefore non infectious and are obtained by a purification procedure of viral coat or capsid elements or the elements may be of fully synthetic origin. A viral or bacterial coat element as used herein should be understood in the broadest sense (which includes coat elements of a phage for example) as a product of a synthetic process such as solid phase peptide synthesis the product of a biological process such as genetic expression of a protein with the help of genetic engineering, a vector and an expression system, or a combination of both processes. Viral and bacterial coat elements can be covalently bound to each other, can be cross-linked with a cross linking agent such as glutaraldehyde or a linker or can be obtained by self assembly of the subunits as for example described for rota virus like particles (31). Self assembly under physiological conditions is a possible method of reconstitution of viral particles. For the purposes of this invention each element or at least one type of elements should contain attachment sides for the hapten or the spacer.

An important class of drugs which may profit from the vaccines and passive immunization of the present invention are AIDS drugs, which include NNRTI drugs (Non Nucleoside Reverse Transcriptase Inhibitors such as Rescriptor, Sustiva, Viramune), NRTI drugs (Nucleoside Reverse Transcriptase Inhibitor drugs such as Ziagen, Trizivir, Epzicom, Videx, Emtriva, Truvada, Epivir, Combivir, Zerit, Viread, AZT, Hivid) PI drugs (Protease inhibitors such as Agenerase, Rayataz, Lexiva, Crixivan, Viracept, Kaletra, Norvir, Invirase), FI drugs (Fusion Inhibitors such as Fuzeon) or any combination thereof. In a combination preparation such as Trizivir, antibodies may be directed of course also against only one drug haptens. Many pharmacologically active drugs with a antiviral potency against AIDS profit from one or a combination of the effects of the interaction of the antibody with the drugs hapten mentioned above. It is therefore a goal of the present invention to obtain one or more of the following beneficial effects due to the interaction of the AIDS drug with specific antibodies: to prolong the half lives of AIDS drugs, to increase therapeutic drug concentration at locations where the drug target can be found, to produce less variations in drug concentration, to obtain better patient compliance due to simpler drug intake schedule, to produce less cost due to lower drug consumption, and to improve the potential toxicity profile of AIDS drugs.

Data in the examples below demonstrate that the in-stream into the brain of a drug molecule as for example nicotine across an intact blood brain barrier in a mammal is very significantly delayed in the initial phase, an effect which breaks the vicious circle between an application of a drug, which may induce dependence and instant gratification and which makes the vaccine so efficient as an anti nicotine vaccine for example. But this observation is only valid for the initial phase of in-stream of drug hapten into the brain. The brain concentrations of radio-labelled nicotine in the brain of the group G2 of example 12 has been measured using the same technique as in the previous examples (radiolabeld intravenously injected nicotine) and has been found to be 35% lower than in the control group GA after 5 minutes, but to be 4% higher (difference not statistically significant due to the small number of animals) than in the control animals after 1 hour. This is an important information for pharmacologically active drugs used in treatments where significant effective drug concentrations in the central nervous system are required over a long period of time as for example for anti-malarial drugs used for treating infestation of the central nervous system or for the application of Multiple Sclerosis or Alzheimer drugs where high effective hapten concentrations in the brain may be desired. It is therefore a goal of the present invention to improve half life, linearity of effective drug concentration, cost effectiveness, ease of application and compliance of patients, for drugs haptens against drugs having a pharmacological activity in the brain.

Acute phases of multiple sclerosis, rheumatoid arthritis, Crohns diseases and other inflammation-based pathologies are characterised by a mechanism where the molecule 4beta1 integrin, which is located on leukocytes in the peripheral blood, binds to vascular cell adhesion molecule-1, which is expressed at high levels in the blood vessels in the CNS. This process allows the 4beta1 integrin population to move across the blood vessel into the brain, where inflammatory protein and leukocyte derived factors acerbate symptoms. 4beta1 integrin specific antagonists have been developed such as D-thioproline-L-tyrosine derivatives (Celltech Chiroscience, Slough, England). These drug haptens have of course very short half-lives, which makes it difficult to maintain high concentrations in the body of a mammal, without continuous replacement. It is therefore a goal of this invention to use antibodies elicited by passive or active immunization against 4 beta1 integrin antagonists in order to significantly improve the half life in vivo, the linearity of effective drug concentration, cost effectiveness, ease of application and patient compliance of the 4 beta1 integrin antagonist.

Materials and Methods:

The large majority of protocols used for the preparation of proteins, purification of proteins, preparation and evaluation of specific antibodies with protocols such as RIA or ELISA are published and we would like to include the following books as references: Ausubel, F. M. Short protocols in molecular biology: a compendium of methods from Current protocols in molecular biology (Wiley, [Hoboken, N.J.], 2002), Weir, D. M. Weir's handbook of experimental immunology (Blackwell Science, Cambridge, Mass., USA, 1996) as well as the instruction book for some of the most used cross spacers and conditions for their use Instructions for NHS-Esters-Maleimide-Crosslinkers by Pierce Biotechnology Inc (Rockford Ill., USA)

Synthesis of an anti-nicotine vaccine: the first coupling strategies are due to Van Vunakis and Langnone (2, 37) which describe the preparation of O-succinyl-3′-hydroxymethyl-nicotine which they use for Radio Immune Assay applications. This preparation shows a low cross-reactivity with cotinine and other metabolites or analogs. This coupling chemistry is in our experience surprisingly resistant in vivo in mice against any hydrolysis or enzymatic degradation as shown below by a long lasting persistence of high anti nicotine antibody titres. Abad et al report on the synthesis of a the nicotine hapten 3′-(hydroxymethyl)-nicotine hemisuccinate (38) and its application to assays. Nicotine haptens linking via position 6 or 1 are described by Castro et al. as well as different other groups authors (39-42). Janda et al. describes another variation attaching to the 1′-position of nicotine (43). Many variations of coupling methods for nicotine have been described in the patent literature (U.S. Pat. No. 5,876,727, U.S. Pat. No. 6,232,082).

Immunization Protocols: An emphasis on all immunization protocols is on avoidance of B-cell or T-cell tolerance and the dose range between 10 and 100 micrograms is therefore chosen. Female Balb C (Harlan) mice, 7 weeks of age are used for experiments if not indicated otherwise. Immunizations are performed by subcutaneous (s.c) injection at the base of the tail of typically 30 microgram of conjugate (nicotine coupled to the carrier protein) in PBS (Phosphate Buffered Saline) together with 1 mg of Alum as (Alu-Gel S, Serva, Switzerland), in a total volume of 60-100 microliter (for mice). Depending on the protocol, the animals are boosted at two- to four week intervals with the same amount of antigen in adjuvant by the same route.

For intranasal (i.n.) immunizations, animals under light anaesthesia are instilled in both nostrils with 5 microliters of conjugate per nostril without adjuvant with the help of a micropipette. Total doses of 3, 10 and 30 microgram of the hapten conjugate in PBS are applied per mouse. One control group receives 30 microgram of the carrier compound only. Two booster instillations are provided on days 7 and 15 post immunization. Saliva is harvested on day 22 and blood on day 29. Anti-nicotine IgA antibodies is tested in the saliva, and IgA and IgG antibodies in the serum. Blood is recovered by tail bleeding

Osmotic pump for dispensing a hapten as for example nicotine: Miniature Alzet osmotic pumps (Alza corporation, USA) model 2004 are implanted into mice subcutaneously on the back of the animal. The pump has a pumping rate of 0.25 microliter per hour and nicotine is delivered during 4 weeks. The administered dose is 1.5 mg/kg/day for a mouse of 20 g, which is estimated to correspond to the nicotine per weight equivalent absorbed by a person weighting 70 kg, smoking 5 packages a day and absorbing 1 mg nicotine per cigarette. The proper functioning of the pump is checked by a RIA assay (RIA nicotine metabolite kit, KCDTDI, Diagnostic Product corporation, USA) measuring cotinine in serum and urine.

Determination of the avidity constant of polyclonal anti nicotine antibodies: The method of R. Muller is used for avidity measurements (44). This classical method is based on a competitive RIA (Radio Immune Assay). The serum dilution, which induces 50% inhibition of the binding of 3H nicotine (N-Methyl-3H nicotine, Sigma No N3876) is determined and the affinity constant calculated by competition with unlabeled nicotine, ((−)-nicotine-(N-Methyl), Sigma No N3876).

Challenge with radioactive nicotine: The rationale for the calculation of the nicotine equivalent of two cigarettes in mice is as follows: a smoker of 75 kg smoking a cigarette absorbs about 1 mg of nicotine. A mouse weights about 20 g and the corresponding quantity per weight is therefore about 300 ng for a cigarette or 600 ng for 2 cigarettes. For practical purposes 597 ng of non labeled nicotine and 3 ng of tritiated nicotine are injected into the tail vein.

Assay of tritium labelled nicotine in the brain and blood: The animals are sacrificed exactly 5 minutes after the injection of nicotine into the tail vein. The blood and the brain are isolated and the brain is digested for 24 hours at 37 degrees in tissue solubilizer solution (Serva, Switzerland). 100 microliters serum plus 2 ml scintillation fluid are used for the nicotine determination in the blood and 200 microliter brain digest plus 2 ml scintillation fluid are used for the nicotine determination in the brain. The radioactivity measured in the brain is corrected for the amount of blood present in the brain, considering that 100 g of brain tissue contain approximately 3 ml of blood (45)

ELISA assay: A standard sandwich ELISA assay is used to measure anti nicotine antibodies. The wells are coated overnight with a nicotine BSA (Bovine Serum Albumin) conjugate in a Carbonate-Bicarbonate buffer, pH 9.6, washed, incubated with blocking buffer (PBS-Tween 200, 5% fat-free powdered milk, 30 minutes, 37° C.) and extensively washed. Test are performed by dilution of the antiserum in PBS buffer, distribution in the wells, incubation (1 h, 37 degree C.), washing and addition of a suitable peroxidase coupled anti-antibody. After washing, the peroxidase substrate OPD and hydrogen peroxide are added as required and the reaction stopped after colour development by addition of H2SO4 before reading the OD at 492 nm in an ELISA reader.

### EXAMPLES

### Example 1

The conjugates based on nicotine haptens are one of the most important applications of the present invention. This example demonstrates the derivatization of nicotine and synthesis of a succinimide ester for use in a coupling procedure. The following experiments demonstrate the synthesis of conjugates based on classical carrier molecules such as nicotine-tetanus toxoid, nicotine-BSA, nicotine-cholera toxin B and three preparations based on a synthetic carrier compound being composed of one or more types of similar elements, where at least one type of element has a functional group serving as a binding site for a hapten. The potential to elicit specific antibodies and the protective effects of classical conjugates is then compared with the protective potential of novel conjugates

Synthesis of a Nicotine-Succinimide Ester:

This example describes the preparation of the ester (+−)-trans-mono(1-methyl-2pyridin-3-yl-pyrrolidin-3-ylmethyl) of Nicotine succinimide acid as well as the preparation and conjugation of nicotine-hydroxy-succinimide ester (Nic-O-Su) to different carrier compounds. The use of the activated ester allows for a specific coupling between an amino- and a carboxy group. The method is an improvement of the strategy originally developed by Langone and van Vunakis (2, 37). The following protocol should be understood as an example and not be limiting. The man skilled in the art, knows how to substitute a succinimide ester by another linker or spacer and further attachment sites for the coupling of nicotine have been in multiple variations described.

a) Esterification: 1.5 equivalent of SOCl₂ is added one drop at a time to a suspension of (+−)-trans-4-carboxycotinine in cold methanol kept in an ice bath. The suspension is after 2 minutes completely dissolved and is left for 2.5 hours at ambient temperature. An excess of HCl formed is eliminated under reflux for 1 hour and the pH is then adjusted to 8 with 10% NaOH. Ethyl acetate is used for extraction and after its elimination a product of 96.7% purity is obtained. Recristallization of 1.2 grams gives a yield of 91% and the product is confirmed with ¹H-RMN at 400 MHz, CDCl₃.

b) Reduction: the gamma-lactame of the methyl-ester is reduced to alcohol. The original procedure using 4 equivalent LiAlH₄ over-night in ether in an inert gas (nitrogen) provides a yield of less than 10%, which is significantly enhanced using THF and doubling the molarity of LiAlH₄. The product is purified with silica chromatography yielding 40 to 60% of the initial weight. The use of NaBH₄ instead of LiAlH₄ provides a comparable result.

c) Condensation of the succinic anhydride: the reaction is performed in 1.5 equivalent succinic anhydride in dichloromethane or dimethylformamide under reflux for 4.5 hours. A SePPack cartridge is used for extraction after purification with a silica column. The product is verified by ¹H-RMN and the yield of this step is 68%.

d) Synthesis of the activated nicotine hapten: the synthesis of nicotine-hydroxy-succinimide ester (Nic-O-Su) is performed in 1.1 equivalent N,N-dicyclohexylcarbodiimide, 1.1 equivalent N-hydroxysuccinimide leading to a 74% yield of this step.

### Example 2

The example describes the conjugation of Nic-O-Su to cholera toxin B Berna. This toxin preparation has been purified from culture derived cholera toxin by the vaccine producer. A second batch of Nic-O-Su to cholera toxin B is produced using recombinant cholera toxin B (rCTB) obtained from SBL vaccines, Stockholm, Sweden. The degree of substitution of nicotine per subunit of the B toxin is in the same range as with the non recombinant toxin (4-5 haptens per subunit) as verified by MALDI-TOF and UV spectroscopy.

Coupling of Nic-O-Su to Cholera Toxin Berna (Berna Serum und Impfinstitut, Bern, Switzerland). 320 equivalent of the activated ester and 320 equivalent of diisopropylethylamine are dissolved in 4 ml H₂O at pH 7.08, room temperature and under agitation by a magnetic stirrer for 1 hour. A first purification is performed with reverse phase High Pressure Chromatography (RP-HPLC) and a second purification step with a dialysis membrane (Pierce Chemicals) having a molecular exclusion limit of 10 000 Dalton is performed with 5 changes of the phosphate buffer having a neutral pH. The product is lyophilized over night. Analysis of the conjugate using UV spectroscopy, RP-HPLC and MALDI-TOF mass spectroscopy (Matrix Assisted Laser Desorption Ionisation-Time Of Flight) shows an average degree of substitution of 4.1 nicotine haptens per cholera Toxin B subunit. The average molecular weight of a subunit is around 12 000 Dalton depending on the degree of substitution. Most of the product is in monomeric form, but multimers can be discriminated on the MALDI-TOF spectrogram.

### Example 3

This example describes the coupling of Nic-O-Su to 2 different synthetic carrier compounds being composed of one or more types of similar elements, where at least one type of element has a functional group serving as a binding site for a hapten. This type of carrier compound is very cheap, can be optimized concerning the degree of substitution and resistance to enzymatic breakdown in vivo and is chemically well defined and uniform, which may speed up regulatory approval and facilitate production and quality control.

Coupling of Nic-O-Su to poly-L-Lysine having a molecular weight range of 150-300 kDa and poly-L-(Ala, Lys) with a molecular weight range of 20-50 kDa (both are purchased from Sigma): 60 mg poly-L-lysine and 10 mg poly-L-(Ala, Lys) are dissolved in a phosphate buffer at a pH of 7.21 respectively pH of 6.84 and 400 equivalent of the activated ester of the hapten are added to each carrier compound. The product is left for 1 hour at 4 degrees and purified by ultra filtration, lyophilized and the degree of substitution semi-quantitatively determined by UV spectroscopy.

### Example 4

Virus like particles are produced in many forms and techniques for production of bulk quantities have been developed.

Coupling of a Nic-O-Su to virus like particles: Nic-O-Su is shipped to Cytos AG, Zürich under argon for coupling to virus like particles with instructions similar as the protocol above.

The animal data in the following two examples are obtained from animals immunized with this virus like particle nicotine conjugate (virus like particles furnished by Cytos AG, immunizations performed by Cytos AG) and assays performed by the inventors.

### Example 5

This example reports results of a Nicotine-RIA inhibition assay (ammonium sulphate precipitation assay) with serum of Nic-O-Su virus like particles immunized mice and rabbits There is a good correlation between results obtained in this assay and the protective effect of the antibodies in an in vivo mice model, where one measures the initial phase of the instream of radiolabeled nicotine in mice vaccinated against nicotine (46):

The protocol according to Muller (44) which measures avidity and specificity of the elicited antibodies is used. The following materials and conditions are used: 50 microgram of tritiated nicotine with a activity of 0.0156 micro curie, 50 microgram serum and 100 microliters RIA buffer are incubated for 2 hours at room temperature 200 microliter of ammonium sulphate is used for precipitation, the suspension is centrifuged after 10 minutes at room temperature for 3 minutes at 13 000 rpm. 200 microliters of the supernatant in 2 ml scintillation liquid are used for measurement of radioactivity:

In Table 1, the letters C1-C3 designates the mean value of mice 1 to 3 of group C, d designates the day of bleeding after the first immunization, the numbers represent the counts per minute (cpm) in the scintillation counter and the resulting percentage value of precipitated nicotine.

TABLE 1

| serum | dilution 1:2 | dilution 1:4 | dilution 1:2 | dilution 1:4 |
|---|---|---|---|---|
| D0, neg.control | 8412 | not done | 0 | not done |
| pos. control | 2263 | not done | 73% | not done |
| mice A1-5, | 5644 | 7050 | 30% | 16.20% |
| d 45 | | | | |
| mice A1.5, | 6140 | 6880 | 27% | 18.20% |
| d 60 | | | | |
| mice A6-10, | 4971 | 6261 | 41% | 25.60% |
| d45 | | | | |
| mice A6-10, | 3939 | 5749 | 53% | 31.60% |
| d 55 | | | | |
| rabbit C1-3 | 4887 | 6687 | 42% | 20.50% |
| d42 | | | | |
| rabbit C1-3 | 5250 | 7006 | 37.50% | 16.50% |
| d70 | | | | |

Those results in Table 1 indicate significantly higher precipitation values as obtained in a previous series of the animals bleeded at day 22 (detailed data no shown) giving values for the percentage of precipitated nicotine between 15.5 and 24.5% at a 1:2 dilution and 5.5-19% at a 1:4 dilution.

### Example 6

This example reports results of an in vivo nicotine challenge study of a batch of mice of which some are previously vaccinated with Nic-O-Su virus like particles. This study is a blind study in the sense that the previous immunization record is not known to the person performing the experiment.

One micro curie of tritiated nicotine (corresponding to 1 512 419 cpm and 2.33 nanogram of nicotine) is intravenously injected in 150 microliters of PBS (Phosphate Buffered Saline) at neutral pH and the mice are sacrificed after 5 minutes. Blood is taken from the inferior vena cava. The brain is taken in an Eppendorf tube. 2 ml of tissue solubilizer fluid are added (Biolute-S, Serva Electrophoresis Gmbh, Germany) and digestion is performed at 40 degrees for 48 hours. For scintillation counting 100 microliter of serum respectively 200 microliter of the brain digest are added to 2 ml of scintillation fluid.

TABLE 2

| | | Radio- | | Brain weight | Blood |
|---|---|---|---|---|---|
| | mouse | ativity | Brain | in 200 μl of | volume in |
| mouse | weight | in serum | weight | brain digest | 200 μl of |
| label | in (g) | (cpm) | (mg) | (mg) | brain digest |
| A1 | 24.3 | 12063 | 447 | 44.7 | 1.34 |
| A2 | 25 | 9641 | 461 | 46.1 | 1.38 |
| A5 | 21.5 | 6789 | 444 | 44.4 | 1.33 |
| A6 | 25.1 | 14043 | 463 | 46.3 | 1.39 |
| A9 | 22.9 | 11995 | 453 | 45.3 | 1.36 |
| A10 | 27.8 | 10292 | 477 | 47.7 | 1.43 |
| B1 | 18.2 | 7084 | 443 | 44.3 | 1.33 |
| B2 | 20.8 | 4625 | 452 | 45.2 | 1.36 |
| B3 | 18 | 6761 | 462 | 46.2 | 1.39 |

In Table 2 the weight of the mouse is given in grams, the serum cpm correspond to the radioactivity (thereafter activity) measured in 100 microliter, the brain weight is measured in milligrams, the fraction of brain in 200 microliter is given in microliter and the blood volume contained in 200 microliter of brain is given in microliter. The calculations performed are based on the estimation that 100 gram of brain contains 3 ml of blood. (45). The data of this table are used to substract nicotine which is contained in the blood of the brain, but which is separated from the receptors in the brain by the blood brain barrier. (This cumbersome procedure is necessary to differentiate nicotine directly in the brain which can potentially reach receptors in the nucleus acumbens from nicotine inside the blood vessels of the brain, which is bound to antibodies and can not reach the brain, because the antibodies can not cross the blood brain barrier).

TABLE 3

| | Total | Radioactivity | Final |
|---|---|---|---|
| Mouse | radioactivity | in brain blood | radioactivity |
| label | in brain (cpm) | fraction (cpm) | in brain (cpm) |
| A1 | 2297 | 162 | 2135 |
| A2 | 2262 | 133 | 2489 |
| A5 | 547 | 90 | 457 |
| A6 | 1585 | 195 | 1390 |
| A9 | 1450 | 163 | 1287 |
| A10 | 1724 | 147 | 1577 |
| B1 | 4859 | 94 | 4765 |
| B2 | 3285 | 63 | 3222 |
| B3 | 4987 | 941 | 4893 |

Table 3 shows total radioactivity of the brain (including blood), activity calculated to be due to blood contained within the brain, and the brain activity with the activity due to blood circulating in the brain deducted (last column).

TABLE 4

| | Nicotine | Nicotine | |
|---|---|---|---|
| Mouse | concentration | concentration | ratio |
| label | in serum (pg/ml) | in brain (pg/ml) | brain/serum |
| A1 | 185.8 | 74 | 0.4 |
| A2 | 148.5 | 83 | 0.56 |
| A5 | 104 | 16 | 0.15 |
| A6 | 216 | 46 | 0.21 |
| A9 | 184 | 44 | 0.24 |
| A10 | 158 | 51 | 0.32 |
| B1 | 109 | 165 | 1.52 |
| B2 | 71 | 166 | 1.54 |
| B3 | 104 | 110 | 1.6 |

Table 4 represents values of nicotine in picogram per millilitre in serum and brain and the brain to serum ratio of the nicotine concentration is noted in the last column. It is clear, that the animals A1 to A6 present less nicotine in their brain and profit therefore from a protective effect.

### Example 7

This example evaluates different carriers with different adjuvant, different immunization methods at two different time intervals in an ELISA assay and demonstrates the influence of those parameters on the capacity of the conjugate to elicit antibodies against nicotine.

TABLE 5

| | groupe code | | | |
|---|---|---|---|---|
| | D0 | D4 | D5 | D6 |
| conjugate | no conj. | nic-rCTB | nic-CTB Berna | nic-rCTB |
| number anim | 6 | 3 | 3 | 3 |
| Adjuvant | not | none | none | Montan.ISA51 |
| | applicable | | | |
| type of immun | not | s.c. | s.c | s.c. |
| | applicable | | | |
| ELISA d 30 | 0 | 1:800 | 1:4173 | 1:10490 |
| ELISA d 60 | 0 | 1:1849 | 1:5501 | 1:8359 |

| | groupe code | | | |
|---|---|---|---|---|
| | D7 | D8 | D9 | D10 |
| conjugate | nic-rCTB | nic-rCTB | nic-rCTB | nic-CTB |
| | | | | Sigma |
| number anim | 3 | 3 | 3 | 3 |
| Adjuvant | Montan.ISA720 | CpG + alum | CpG | none |
| type of immun | s.c | s.c | nasal | nasal |
| ELISA d 30 | 1:5274 | not done | not done | not done |
| ELISA d 60 | 1:9081 | 1:5179 | 1:5891 | 1:11565 |

The following abbreviations are used in Table 5: nic-rCTB is a nicotine conjugate using a recombinant cholera toxin B carrier, nic-CTB Berna uses the purified cholera toxin obtained from Berna, Switzerland, nic-CTB Sigma uses a purified cholera toxin from Sigma, alum means aluminium hydroxide adjuvant from Berna, Switzerland, Motanide ISA-51 and Montanide ISA-720 are adjuvant obtained from Seppic SA, France, CpG is a polynucleodide based adjuvant. d 30 means that the serum was taken 30 days after the first immunization. 30 micrograms of conjugate are used for immunizations.

The cut off between a positive and a negative ELISA result has been calculated as 3 standard deviations of the optical density measured as background value. The titres of dilution in the above table have then been obtained by diluting the serum sample from mice at days 30 and 60 and calculating the dilution factor which coincides with the cut off value between positive and negative value by interpolation.

An ammonium sulphate precipitation RIA was performed with all samples with the sample D6 showing a 50% precipitation of radio labelled nicotine. Nasal stimulation alone (D9) is capable to elicit precipitating antibodies, but to a lesser degree than s.c. immunization. We have shown in published work a cumulative effect if intra-nasal and subcutaneous immunizations are combined sequentially (s.c then i.n) for an antinicotine vaccine (46). Many teachings concerning adjuvant and route of immunization as well as schedule of immunization are widely applicable as far as carrier compounds are concerned. The efficiency of intranasal nicotine immunization studies is due to significant stimulation of IgA anti nicotine antibodies as shown below.

### Example 8

The following results have been obtained with a Nic-O-Su-Cholera Toxin B conjugates (46). The hapten spacer compound of a conjugate defines the specificity of the conjugate and the obtained from the Nic-O-Su-Cholera Toxin B conjugates can be generalised to include the Nic-O-Su-based conjugates having other carrier components, especially if the attachment site is the amino group of the side chain of a lysine.

Immunologic cross reactivity of anti-nicotine antibodies: The competitive RIA assay as described is used to measure immunologic cross-reactivity with other biological compounds, which are related to nicotine. The following cross-reactivities have been found: cotinine: 1.1%, nornicotine: 4%, trans-4-cotininecarboxylic acid: 0.19%, acetylcholine: 0.05%, nicotinic acid: 0.09%. The low cross-reactivity of acethylcholine is comparable to background noise of the assay system.

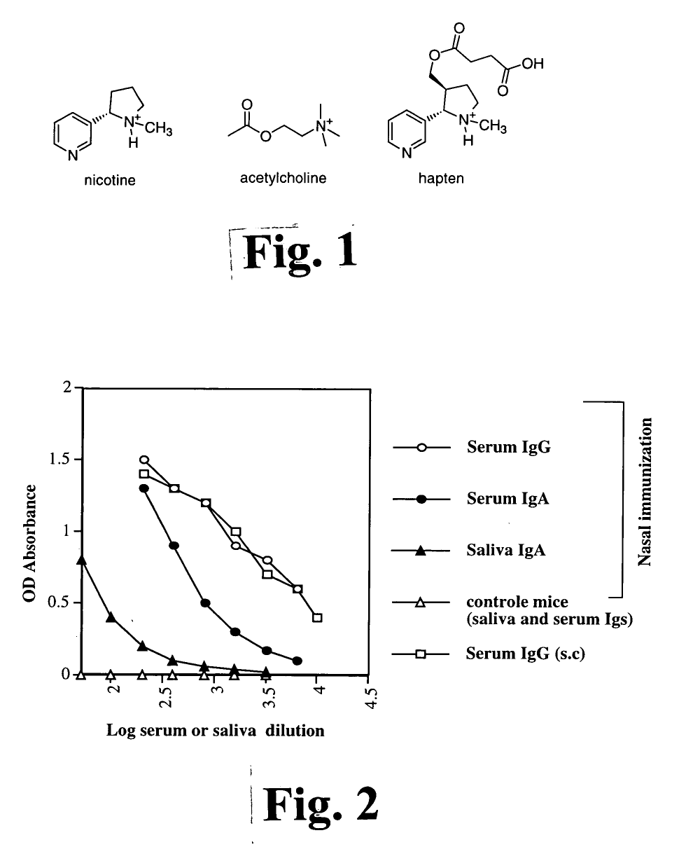

FIG. 1 shows the close structural relationship as well as the similarity in electric charge distribution between the nicotine molecule, acetylcholine and the nicotine hapten spacer compound as described in this invention (trans-3′-Succinylmethylnicotine).

ELISA results after intranasal and subcutaneous challenge: Cholera Toxin B induces significant IgA titres, when given i.n. The IgA antibodies can be detected in saliva as well as in the serum. The figure below shows IgG and IgA ELISA results of saliva and serum with nicotine BSA coated to the solid phase. Total closes of 30 microgram of the nicotine CTB Berna conjugate are applied per mouse in PBS to both nostrils whereas a control group receives 30 microgram of cholera toxin only. Two booster instillations are provided on days 7 and 15 post immunization and saliva is harvested on day 22 and blood on day 29. Immunization with the nicotine CTB Berna conjugate induces significant anti nicotine IgA titres, when given i.n. The IgA antibodies can be detected in the saliva as well as in the serum. FIG. 2 shows the results of IgA and IgG measurements in both saliva and serum as determined by ELISA using a nicotine BSA conjugate coated to the solid phase. Each data point presents the result of pooled serum of five animals.

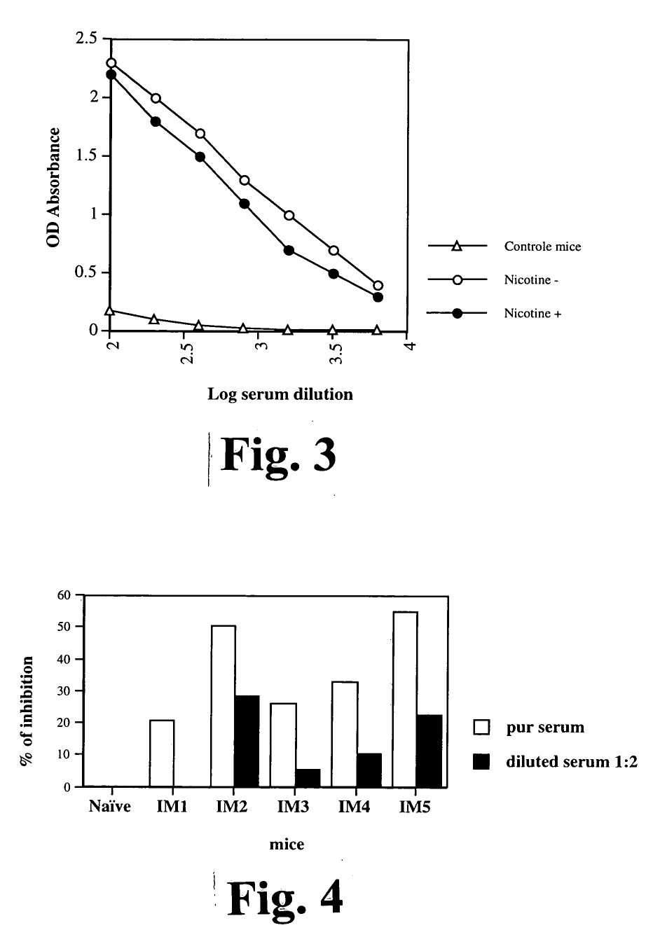

ELISA results in mice with an implanted nicotine pump: FIG. 3 represents the optical density (OD) readings of serial dilutions of serum evaluated in a sandwich ELISA assay with nicotine-albumin coated to the solid phase, measuring total anti-nicotine antibodies with an enzyme labelled second anti antibody. The serum is taken 5 weeks after the initial immunization. The group “control mice” represent negative controls, the group “Nicotine −” a group which was vaccinated with nicotine rCTB conjugate and the group “Nicotine +” represents a group of mice vaccinated under the same conditions (nicotine rCTB, 2×s.c. with Alum, 2 weeks interval), which have in addition the nicotine pump implanted, dispensing during 4 weeks the nicotine equivalent of 5 packages of cigarettes a day (1.5 mg/kg/25 hours). There is no significant difference in antibody titres between the two groups (each data point represents the pooled sera of 5 mice).

Determination of the binding capacity of antibodies to nicotine by a soluble RIA: FIG. 4 demonstrates the development of anti nicotine specific antibodies in mice as measured by the precipitation of the anti nicotine-³H(−)-nicotine complexes by ammonium sulphate in a RIA after different intranasal (i. n.) and/or subcutaneous (s. c) immunisation schedules with 30 microgram of the conjugate (nicotine CTB Berna). The interval between subsequent immunizations is 2 weeks (each data point corresponds to pooled serum of 6 animals). IM1: 3×i.n; IM2: 3×s.c; IM3: 3×i. n+s.c; IM4: 3×i. n+2×s.c; IM5: 3×s.c+2×i.n. The animal IM3 receive i.n and s.c. challenges at the same time, the animals IM4 and IM5 receive the two types of immunization sequentially.

Distribution in the serum and the brain of tritium labelled nicotine bolus: FIG. 5 shows the bolus injected into the tail vein corresponding to the equivalent of 2 cigarettes (600 ng in a mice of 20 g) in mice sacrificed five minutes after injection. As one would expect, a significant amount of the nicotine is bound in the serum of the vaccinated animals as compared to the naïve animal, but less than 10% of the dose can be found in the brain. (IM1: 3×i.n., IM2 3×s.c., serum of 5 animals is pooled for each data point).

### Example 9

This example describes the coupling of pharmacologically active haptens with at least one amino group to a polypeptide carrier using glutaraldehyde for coupling. The following haptens are chosen for coupling: Trimethoprime, Primaquine, Pyrimethamine, Sulfadiazine, Dapson (diamionodiphenylsulfone). Dapsone has antileprotic activity, Trimethoprime is an antibiotic with a large spectrum often used at low dosage for chronic infections such as urinary Escherichia Coli infections, Primaquine and the other drug haptens have ant malarial activity. The following conjugates in conjunction with those haptens can be used for therapeutic purposes, where the prolonged half life of the drug hapten after vaccination allows a protective effect in conjunction with a significantly diminished intake of the medications. Glutaraldehyde coupling and polypeptide carrier compounds are particularly cheap and allow the manufacturing of vaccines which can be manufactured and financed by less developed countries. On the other hand is the final product partially cross linked and not homogenous.

Glutaraldehyde coupling: a) 10 mg of a polypeptide with a molecular weight of at least 100 000 Da and at least 5 lysine amino acids per peptide are dissolved in 2 ml of a 0.1 M borate buffer at pH 10. A 100 times molar excess of the hapten with at least one amino binding site is added slowly under constant stirring with a magnetic stirrer. b) 1 ml of a 0.3% solution of glutaraldehyde (electron microscopy grade, Sigma) is added drop wise under stirring and the reaction mixture is left at room temperature for 30 minutes. c) The non occupied binding sites of glutaraldehyde are saturated with 0.25 ml of 1 M glycine dissolved in the conjugation buffer and the conjugate is dialysed against a 0.1 M borate buffer using a dialysis membrane with an exclusion limit of 10 000 Da, pH 7.6 and frequent buffer exchange in the cold room overnight.

The man skilled in the art knows how to substitute a bifunctional spacer in place of glutaraldehyde and how to minimize cross linking by using 2 step procedures, where the spacer is linked to the hapten and the carrier elements in two separate sequential steps.

### Example 10

We have mentioned above, that the half life of nicotine in vivo in mice increases by a factor of 10 or more if the animal has been efficiently vaccinated against nicotine (29). We have also mentioned above published data by Smith at al. (35), which show that the half life of the hapten in the digoxin model is significantly increased in vaccinated animals as compared to data from the nicotine hapten model, showing in the digoxin hapten model after one year in the serum of vaccinated animals a concentration of the hapten, which is reached already after 12 hours in non vaccinated animals, which corresponds to a factor of 700.

A pill with a pharmacologically active hapten which has to be taken once a week, month or even a year would represent a significant improvement over a pill, which has to be taken once every 12 hours and it seems worthwhile to examine the reasons for the differences in the above model in detail.

There are 3 crucial parameters in vivo, which govern retention of the hapten by specific antibodies.

a) the in vivo half life of the epitope against which the antibody is directed: haptenic epitopes which are rapidly altered by metabolism of the hapten are no more retained by the antibody because their steric configuration is altered and the key fits no more into the lock.

b) antibody avidity.

c) the distribution in vivo of the hapten (distribution volumes, distribution among different tissues). A hapten interacts only at places with the antibodies, where antibody can be found.

The persistence of the epitope of the hapten in its original form in vivo as mentioned under a) is a “”conditio sine qua non” for a long lasting effect of the antibodies on the hapten. One may argue that this is not necessary the case because the hapten may be shielded inside an immune complex from any metabolic interaction altering the epitope. But there is experimental evidence that immune complexes involving haptens like nicotine, digoxin or morphine are continuously build and deconstructed as shown by an in vitro and an in vivo argument:

a) non labelled morphine, given to N-methly-3H morphine-antibody complexes in a dialysis chamber having a membrane with an exclusion limit of 10 000 Da is able to displace the labelled hapten from the antibody as indicated by lower radioactivity inside the chamber (details below)

b) Berkowitz and colleagues inject tritiated dihydromorphine in mice vaccinated against morphine and and report (page 1023, third paragraph): “However, morphine can displace the dihydromorphine from the antibody in vivo since the plasma levels return to control values if (original text: of) morphine is given one hour after dihydromorphine.” (47)

Displacement of radio labelled morphine bound to specific antibodies in a dialysis chamber by incubation with different amounts of non labelled nicotine: 5 duplicate samples of 0.1 ml serum of mice vaccinated against morphine are diluted each with 0.9 ml PBS, pH 7.2, containing 2 microgram of morphine labelled by addition of tritiated morphine (N-methy-3H morphine, New England Nuclear, Boston) and incubated at room temperature for 60 minutes in a spinfuge tube having a membrane with an exclusion limit of 10 000 Da. The samples are centrifuged and washed 3 times with PBS buffer and reconstituted with 1 ml of PBS. 100 microliter of the suspension of each sample are diluted in 2 ml scintillation liquid and the morphine binding capacity is calculated (mean value of samples: 0.6 microgram morphine per ml mouse serum). 100 microliter non labelled morphine is added containing 0, 1, 2, 3, 4 microgram of morphine, and incubation (30 min, room temperature), centrifugation, washing, reconstitution of the sample as well as scintillation counting are repeated as above. The results show a replacement of the labelled morphine by non labelled morphine except in the negative control sample.

### Example 11

It is well known, that conjugates are able to induce immune responses lasting from duration of a couple of month to a life long protection. The original work of Freund used oily emulsions of bacterial cell walls (48), where the original vaccine respectively droplets of the emulsion could be found on histological sections taken from the site of injection 1 year after vaccination. It is clear from example 10 that the in vivo half-life of the epitope of the hapten is a requirement for a long lasting immune answer, and that in situ alteration or destruction of the epitope eliciting the immune answer will be an important parameter as far as the duration of induction of immune response of a vaccine. On the other hand, if a long lasting immune response is induced, it can be deducted, that he conjugate is resistant to rapid alteration by the conditions encountered in vivo at the place of injection.

A group of 10 mice vaccinated under conditions described in experiment 8 with the Cholera toxin B (Berna) at the base of the tail (subcutaneously, Alum as adjuvant) are observed for a duration of 11 month and ELISA assays are performed at regular intervals. The mean ELISA titer in the serum of the animals after duration of 11 month is still above 40% of the peak values measured during this period. The conclusion is made, that the epitope inducing a specific immune response of the nicotine hapten used in the conjugate is resistant to alteration in vivo and should be considered as a candidate for development of a long lasting vaccine against nicotine.

### Example 12

This example shows the fate of radio-labelled nicotine in mice which have been immunized with the Nic-O-Su-carrier protein vaccine. We have demonstrated in example 8, that this nicotine hapten-spacer compound demonstrates only a very low cross reactivity with some of the most important metabolites of nicotine.

The liver is the most important place of nicotine metabolism (all the following compiled data are for humans, but data for mice are not fundamentally different), where 70 to 80% of nicotine is converted to cotinine in a 2 step procedure by a cytochrome P450 system (CYP2A6 and CYP2B6) and aldehyde oxidase. 4-7% of nicotine is transformed in the liver to nicotine N′-oxide by a flavin containing monooxygenase 3. Another metabolic pathway of nicotine occurring in the liver and accounting for 3-5% of metabolized nicotine is nicotine glucuronidation leading to an N-quaternary glucuronide, a reaction which is catalyzed by uridine diphosphate-glucuronosyltransferase. Finally, a small percentage of nicotine is transformed in the liver to nor-nicotine, a process which is thought in rabbits to be mediated by the cytochrome P 450 system (the data for this paragraph have been compiled from the Pharmagenetics and Pharmagenomics Knowledge data base).

In view of the fact, that a) the liver is the most important place of metabolism of nicotine and b) that the liver is highly vascularized, we would expect in mice vaccinated against nicotine an initial increase of the nicotine concentration in the blood, corresponding to the immune-concentration phase, where the antibody binds the hapten. We would then expect after a latency corresponding to the time it takes to chemically alter the nicotine in the liver a decrease in the radiolabeled nicotine concentration in the blood, because the metabolites escape binding to the specific antibodies based on low cross reactivity. The data below taken from vaccinated mice observed during the first 60 minutes show this initial 2 phase pattern of retention of the radiolabelled nicotine hapten. One would like to confirm the interpretation of this data by a simultaneous HPLC- or mass-spectrographic analysis of the nicotine metabolites in the blood, as well of cross reactivity data of the antibodies elicited by the vaccine with each of those metabolites, information which is at this time outstanding.

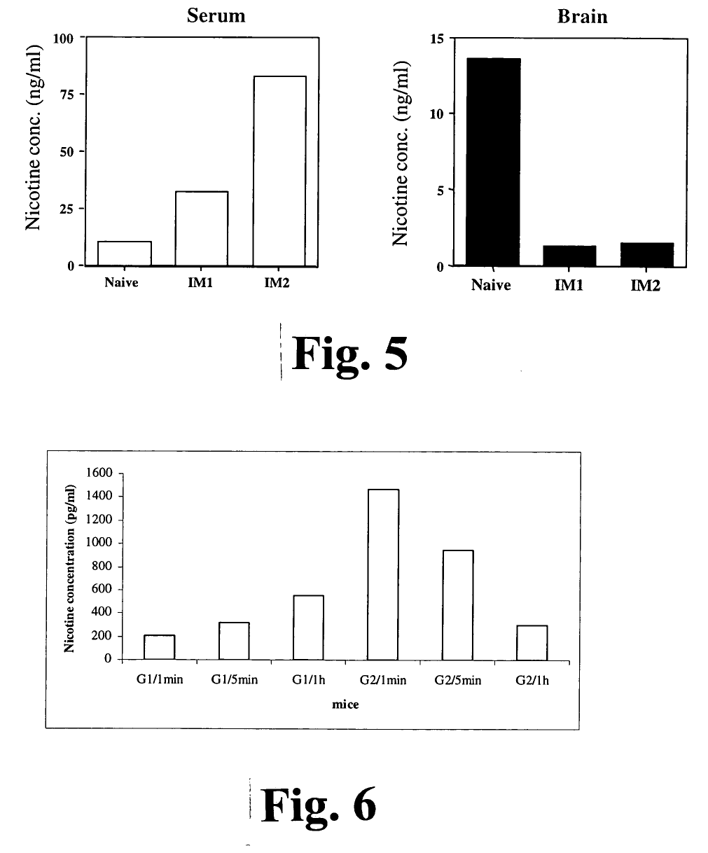

FIG. 6 shows scintillation counter data (cpm) after challenge with radiolabelled nicotine (1 minute, 5 minutes, 1 hour) of non immunized control mice (G1, all groups composed of 5 animals), mice immunized by subcutaneous immunization (G2). Injections were made intravenously with the help of an insulin syringe, injecting 100 microliter containing 5 micro curie of nicotine in PBS, corresponding to 11.65 nanogram nicotine and blood samples were taken from the inferior vena cava.
1. S. Spector, C. W. Parker, Science 168, 1347 (Jun. 12, 1970).
2. J. J. Langone, H. B. Gjika, H. Van Vunakis, Biochemistry 12, 5025 (Nov. 20, 1973).
3. S. J. Mule, D. Jukofsky, M. Kogan, A. De Pace, K. Verebey, Clin Chem 23, 796 (May, 1977).
4. D. H. Schmidt, V. P. Butler, Jr., J Clin Invest 50, 866 (April, 1971).
5. B. Berkowitz, S. Spector, Science 178, 1290 (Dec. 22, 1972).
6. K. F. Bonese, B. H. Wainer, F. W. Fitch, R. M. Rothberg, C. R. Schuster, Nature 252, 708 (Dec. 20, 1974).
7. A. Killian, K. Bonese, R. M. Rothberg, B. H. Wainer, C. R. Schuster, Pharmacol Biochem Behav 9, 347 (September, 1978).
8. Strahilevitz. (United States Patent Office, 1977).
9. I. E. Kovalev, O. Polevaia, S. E. Metkalova, L. A. Basharova, M. M. Borisov, Farmakol Toksikol 42, 615 (November-December, 1979).
10. E. H. Cerny. (Switzerland, 1990).
11. D. N. Posnett, H. McGrath, J. P. Tam, J Biol Chem 263, 1719 (Feb. 5, 1988).
12. M. Mutter, R. Hersperger, K. Gubernator, K. Muller, Proteins 5, 13 (1989).
13. G. Tuchscherer, M. Mutter, J Biotechnol 41, 197 (Aug. 31, 1995).
14. C. Nkubana, Diplome d'Etudes superieures en Chimie, Université de Lausanne (1999).
15. C. Henzelin-Nkubana, Doctoral Thesis, Ecole Polytechnique Féderalé de Lausanne (2002).
16. E. H. Cerny. (Switzerland, 1994).
17. E. H. Cerny. (1994).
18. C. J. Murray, A. D. Lopez, Science 274, 740 (Nov. 1, 1996).
19. C. J. Murray, A. D. Lopez, Lancet 349, 1436 (May 17, 1997).
20. C. J. Murray, A. D. Lopez, Epidemiology 10, 594 (September, 1999).
21. T. Cerny, Onkologie 25, 404 (November 2002).
22. G. Kohler, C. Milstein, Nature 256, 495 (Sep. 7, 1975).
23. C. G. Nwosu, P. A. Crooks, Xenobiotica 18, 1361 (ÿ %\□°, 1988).
24. P. A. Crooks, C. S. Godin, W. F. Pool, Med. Sci. Res. 20, 879 (1992, 1992).
25. J. W. Gorrod, P. Jacob, Eds., Analytic determination of nicotine and related compounds and their metabolites (Elsevier Science, 1999), pp. 69-135.
26. B. Benacerraf, E. R. Unanue, Textbook of immunology (Williams & Wilkins, Baltimore, ed. 2nd, 1984), pp. 324.
27. G. Tribbick, A. B. Edmundson, T. J. Mason, H. M. Geysen, Mol Immunol 26, 625 (August, 1989).
28. E. Jensen-jarolim et al., Faseb J 12, 1635 (1998).
29. Y. Hieda, D. E. Keyler, S. Ennifar, A. Fattom, P. R. Pentel, Int J Immunopharmacol 22, 809 (October, 2000).
30. F. Lechner et al., Intervirology 45, 212 (2002).
31. C. M. O'Neal, S. E. Crawford, M. K. Estes, M. E. Conner, J Virol 71, 8707 (December, 1997).
32. T. A. Schmidt, K. Kjeldsen, J Cardiovasc Pharmacol 17, 670 (May, 1991).
33. V. P. Butler, Jr. et al., J Clin Invest 59, 345 (March, 1977).
34. D. H. Schmidt, V. P. Butler, Jr., J Clin Invest 50, 1738 (September, 1971).
35. D. H. Schmidt, B. M. Kaufman, V. P. Butler, Jr., J Exp Med 139, 278 (Mar. 1, 1974).
36. B. Rodu, P. Cole, Nature 370, 184 (Jul. 21, 1994).
37. J. J. Langone, H. Van Vunakis, Methods Enzymol 84, 628 (1982).
38. A. Abad, J. J. Manclus, C. March, A. Montoya, Anal Chem 65, 3227 (Dec. 15, 1993).
39. H. Matsushita, M. Noguchi, E. Tamaki, Biochem Biophys Res Commun 57, 1006 (May 23, 1974).
40. M. Noguchi, H. Matsushita, Y. Tsujino, Biochem Biophys Res Commun 83, 83 (Aug. 14, 1978).
41. A. Castro, I. Prieto, Biochem Biophys Res Commun 67, 583 (Dec. 17, 1975).
42. Med Lett Drugs Ther 39, 77 (Aug. 15, 1997).
43. S. Isomura, P. Wirsching, K. D. Janda, J Org Chem 66, 4115 (Jul. 15, 2001).
44. R. Muller, Methods Enzymol 92, 589 (1983).
45. N. Kaliss, D. Pressman, Proc. Soc. Exp. And Med. 75, 16 (October, 1950).
46. E. H. Cerny et al., Onkologie 25, 406 (2002).
47. B. A. Berkowitz, K. V. Ceretta, S. Spector, Life Sci 15, 1017 (Sep. 1, 1974).
48. J. Freund, Am J Clin Pathol 21, 645 (July, 1951).

We claim:

1. A conjugate for the immunization of mammals which is able to elicit in a mammal antibodies against a given hapten, said conjugate comprising:
a) a synthetic carrier compound being composed of one or more types of similar elements, where at least one type of element has a functional group serving as a binding site for a hapten.
b) at least one hapten chosen from the group of pharmacologically active molecules, said hapten having a number of epitopes not allowing the formation of immune complexes inducing pathological changes in a mammal and being linked preferably by a covalent bond to the site of binding for a hapten of said carrier compound,
c) optionally a spacer compound, which forms a bridge between the carrier compound and the hapten and is preferably linked by a covalent bond to the binding sites of the hapten and the carrier compound.

2. The conjugate of claim 1 eliciting antibodies which are used for passive immunization.

3. The conjugate of claim 1 having a molecular weight in excess of 10 000 Dalton.

4. The conjugate of claim 1 where said carrier compound being composed of a multitude of one or more types of similar elements is composed of a type of elements selected from the group of peptide or protein polymers, sugar polymers, viral or bacterial coat elements or any combination thereof with or without metatranslational changes.

5. The conjugate of claim 1 where said hapten itself has no pharmacological activity but is an immunological mimotope of said hapten, having the capacity to elicit antibodies against said hapten chosen from the group of pharmacologically active molecules.

6. The conjugate of claim 1, wherein the binding site of said carrier compound is an amino group belonging to a lysine residue.

7. The conjugate of claim 1, wherein the site of binding for a hapten is linked to said carrier compound attachment site through a non-peptide bond.

8. The conjugate of claim 1, wherein the binding site of said carrier compound comprises but is not limited to one of the following functional groups: (a) an amino group; (b) a carboxyl group; (c) a sulfhydryl group; (d) a hydroxy group

9. The conjugate of claim 1, wherein the site of binding for said hapten is a functional group selected from but not limited to one of the following functional groups: carboxyl, amine, sulfhydryle, amide, succinimide, maleimide, and aldehyde.

10. The conjugate of claim 1, where the antibodies elicited by said hapten are directed against one or more drugs which may induce dependence.

11. The conjugate of claim 10, where the antibodies elicited by said conjugate are directed against one or more drugs of abuse selected but not limited to the group consisting of: opiates, cocaine, amphetamines, anti depressive drugs, PCP, LSD, nicotine, psycho mimetic drugs or derivatives or metabolites of such drugs.

12. The conjugate of claim 1, wherein said hapten contains at least one epitope against which the antibody response is directed, which is not or only very slowly metabolised in vivo into an epitope having a lower or no avidity for said antibody.

13. The conjugate of claim 12, wherein the hapten induces antibodies against a pharmacologically active hapten, which has an antiviral activity against Human Immunodeficiency Virus.

14. The conjugate of claim 1, wherein the hapten is formed from starting materials eliciting antibodies against L-nicotine, D-nicotine, a racemic mixture of L- and D-nicotine.

15. The conjugate of claim 14, wherein the hapten is formed from starting materials selected from the group consisting of trans-4′-carboxycotinine.

16. The conjugate of claim 14, wherein said conjugate comprises O-succinyl-3′-hydroxymethyl-nicotine conjugated to a peptide polymer or a virus like particle.

17. The conjugate of claim 14 wherein O-succinyl-3′-hydroxymethyl-nicotine as the hapten and spacer compound is bound to a lysine side chain of the carrier compound.

18. The conjugate of claim 14 wherein O-succinyl-3′-hydroxymethyl-nicotine as the hapten and spacer compound comprises the L- and D-enantiomer of nicotine.

19. The conjugate of claim 14, wherein the binding site on the carrier compound is an amide.

20. A galenic formulation for treating or preventing addiction to a drug which may induce dependence, by application of a vaccine or passive immunization comprising the conjugate of claim 14 and an excipient.

21. A galenic formulation of the conjugate of claim 14 for treating or preventing addiction to a drug, which may induce dependence comprising the conjugate and an adjuvant.

22. A method of treating or preventing addiction to a drug which may induce dependence, said method comprising administering to an individual the conjugate of claim 14.

23. A method of treating or preventing addiction to a drug, which may induce dependence, said method comprising administering to an individual by passive immunization an antibody elicited by the conjugate of claim 14.

24. A method of treating an overdose of a drug, which may induce dependence, said method comprising administering to an individual by passive immunization an antibody elicited by the conjugate of claim 14.

25. A method of treating an overdose of a drug, said method comprising administering to an individual by passive immunization an antibody elicited by the conjugate of claim 1.

26. A method of inducing an immune answer to a drug in an animal, said method comprising administering an amount of the conjugate of claim 20 to a mammal, which elicits a maximal efficient antibody response to said drug.

27. The method of claim 26, wherein said conjugate is administered to said animal by a route selected from but not limited to the group consisting of intra-nasally, orally, subcutaneously, trans-dermaly, intra-dermaly, intra-muscularly or intravenously or any combination thereof.

28. The method of claim 14 wherein at least one route of administration is intranasal and the antibodies are directed against cocaine or nicotine.

29. The method of claim 28 involving more than one immunization.

30. The method of claim 29, wherein the immunizations are by the same, or different routes.

31. The method of claim 30 wherein the hapten is nicotine, the carrier compound made of virus like particles and the spacer a succinimide ester.

32. The method of claim 31, where said composition is administered without or together with an adjuvant to said mammal, transdermally, intra-dermally, subcutaneously, intra-nasally, orally, intramuscularly or intravenously.

33. The use of the conjugate of claim 1 for the manufacture of a medication for drug rehabilitation treatment respectively smoking cessation treatment.

34. A method for preventing or treating nicotine addiction and alleviating nicotine withdrawal symptoms at the same time, comprising the step of administering to a patient the conjugate of claim 14 and a nicotine substitution product.

35. The method of claim 34, wherein the nicotine substitution product has a lower dose than products on the market today, because of the accumulation of nicotine in serum of vaccinated mammals by binding to anti nicotine antibodies.

36. The method of claim 34 wherein said vaccine composition is administered intra-nasally, orally, subcutaneously, trans-dermaly, intra-dermaly, intramuscularly or intravenously, and wherein said additional agent is administered orally or via a trans-dermal patch.

37. The method of claim 34 wherein said vaccine composition comprises O-succinyl-3′-hydroxymethyl-nicotine conjugated to a polypeptide with or without metatranslational modifications.

38. The method of claim 34 wherein said vaccine composition comprises O-succinyl-3′-hydroxymethyl-nicotine conjugated to a virus like particle.

39. The method of claim 36 where said additional agent administered orally or via a trans-dermal patch dispenses nicotine in order to overcome withdrawal symptoms.

40. The evaluation of said galenic formulation of claim 20 for diminishing cardiovascular morbidity and mortality in mammals who inhale actively or passively cigarette containing smoke.

41. The conjugate of claim 1 intended for the treatment of AIDS where said hapten is selected but not limited to the group of Non Nucleoside Reverse Transcriptase Inhibitors, Nucleoside Reverse Transcriptase Inhibitors, Protease inhibitors and Fusion Inhibitors or any combination thereof.

42. A galenic formulation including the conjugate of claim 41 and at least one excipient having at least one therapeutically beneficial effect on the pharmacological effectivness of an anti AIDS drug hapten selected from the following group of effects: prolonging the half life in vivo, improving the linearity of effective drug concentration, improving cost effectiveness, improving ease of application and patient compliance.

43. A method of improving treatment with an anti AIDS drug hapten, said method comprising active immunization with the conjugate of claim 41 or passive immunization with a compound having similar avidity and specificity for the anti AIDS drug hapten as antibodies elicited by the conjugate of claim 41.

44. The conjugate of claim 1 where said hapten is selected from the group of drug haptens being used for prevention or therapy of malaria.

45. A galenic formulation including the conjugate of claim 44 and at least one excipient having at least one therapeutically beneficial effect on the pharmacological effectivness of a drug hapten being used for prevention or therapy of malaria selected from the following group of effects: prolonging the half life in vivo, improving the linearity of effective drug concentration, improving cost effectiveness, improving ease of application and patient compliance.

46. A method of improving treatment with a drug hapten being used for prevention or therapy of malaria, said method comprising active immunization with the conjugate of claim 44 or passive immunization with a compound having similar avidity and specificity for the 4beta1 integrin antagonist as antibodies elicited by the conjugate of claim 44.

47. The conjugate of claim 1 where said hapten is selected from the group of 4 beta1 integrin antagonists.

48. A galenic formulation including the conjugate of claim 47 and at least one excipient having at least one therapeutically beneficial effect on the pharmacological effectiveness of 4beta1 integrin antagonists selected from the following group of effects: prolonging the half life in vivo, improving the linearity of effective drug concentration, improving cost effectiveness, improving ease of application and patient compliance.

49. A method of improving treatment with a 4 beta1 integrin antagonists, said method comprising active immunization with the conjugate of claim 47 or passive immunization with a compound having similar avidity and specificity for the 4beta1 integrin antagonist as antibodies elicited by the conjugate of claim 47.
